(19) United States
(12) Patent Application Publication
(10) Pub. No.: US 2021/0330138 A1
(43) Pub. Date: Oct. 28, 2021

(54) Ear Hygienic Implements and Kit

(71) Applicant: Sunny Dukovich, Los Angeles, CA (US)

(72) Inventor: Sunny Dukovich, Los Angeles, CA (US)

(21) Appl. No.: 16/859,642

(22) Filed: Apr. 27, 2020

(51) Int. Cl. A47K 7/04; A46B 5/00; A46B 13/02; A45D 34/04; A45D 40/00; A45D 44/00

(52) U.S. Cl. CPC A47K 7/04; A46B 5/0095; A46B 13/02; A45D 34/042; A45D 2200/1036; A45D 44/00; A46B 2200/10; A45D 2200/1018; A45D 40/00

(57) ABSTRACT

An implement for cleaning ears is shown and described. The implement may include an elongated stem and a foam or bristle flexible cleaning head removably or permanently coupled thereto. Optionally, a second, different head may be provided at an opposed end of the stem. Optionally, the cleaning head may be rotatable or vibrate under electric power. In addition, the cleaning head may be deployable from a stored position in a flexible tube to an extraction position outside of the flexible tube, wherein the mechanism can either be electrical or non-electrical. The implement may be part of a kit also including a container of containing oil foam having oil, soap, and water; a package containing a supply of liquid permeable pads impregnated with antibacterial liquid; and a spray container containing antibacterial liquid. Optionally, the kit also includes a container containing a supply of liquid permeable pads impregnated with an astringent agent.

### FIELD OF THE INVENTION

The present invention relates to personal hygiene, and more particularly, to brushes, sponges, or any cloth swabs for cleaning the ear internally and externally with or without cleaning reagents.

### BACKGROUND OF THE INVENTION

Ears provide an opening into the body which opening is susceptible to infiltration by dirt and pathogens. These may be airborne, transferred by finger, introduced by use of audio objects such as cellular phones and head worn microphones, and introduced inadvertently in the course of washing and showering. In addition, ears are sources of bodily secretions as well, such as wax and sweat.

Currently available implements for ear cleaning, such as cotton fiber tipped swabs, inner ear metal and plastic implements, and rubber and silicone implements entail drawbacks. Swabs may push wax and dirt into the inner ear and trapping bacteria deep within the ear canal. Metal and plastic implements can injure ear tissues. Rubber and silicone implements may be partially ineffective as they cannot engage and transport wax and dirt from the inner ear. Liquid ear cleansing drop products also cannot remove solids, and moreover may be messy to use.

There exists a need for an ear cleaning implement providing additional capabilities.

### SUMMARY OF THE INVENTION

The present invention provides an ear cleaning implement with enhanced ability to trap and extract solid detritus from the ears, compared to many prior art devices. Notably, the implement selectively includes an open cell foam (i.e. sponge), fabric, or bristle lined cleaning head. This construction both enables distribution of cleansing or therapeutic fluids and also can trap solids for extraction. Both the open cell head and the head bearing bristles may be either permanently fixed to a handle, or removably fixed to the handle. In addition, the cleaning head may include stops or flared sections upon the cleaning head to prevent the cleaning head from being inserted too far into the ear canal, thereby preventing any potential injury.

In addition, a variation of the invention is presented wherein a flexible tube mounted on a stem is utilized for insertion of a cleaning head into the ear canal. The flexible tube houses and compresses a cleaning head in an insertion position and is utilized to provide smooth insertion into the inner ear canal without pushing wax and/or dirt in. Once the flexible tube is inserted, the cleaning head is then advanced out of the flexible tube to an extraction position in the ear canal. In the extraction position, the cleaning head expands to allow for the cleaning of the ear canal as cleaning head is pulled out of the ear canal. Manual (non-electric) and mechanical (electric) versions of this variation are presented.

Each style of cleaning head can engage the handle by elastic contraction or alternatively by interengaging spines or teeth on both the cleaning head and the handle. The portion of the handle engaging the cleaning head may be enlarged in transverse dimension(s).

In an option, the cleaning head may be rotatable and vibrate under electric power.

In a further option, the implement may include two cleaning heads, one at each end of the handle. Where two cleaning heads are provided, the two heads may differ in characteristics such as dimensions, proportions, and specific cleaning surface.

In a further option, the implement may be part of a kit including hygienic or therapeutic/cleansing fluids and pliable finger held or wrapped-on textured pads (or wipes), impregnated pads for applying hygienic fluids previously impregnated into the pads. The kit may include a spray dispenser for some therapeutic fluids. The kit may also include a bottle of cleaning liquid, gel, foam, paste, or oil to be used with this implement with or without water.

The present invention provides improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a diagrammatic side cross sectional view of an implement for cleaning ears, according to at least one aspect of the invention;

FIG. 2 is a diagrammatic side cross sectional view of a specific form of a component seen at the top of FIG. 1;

FIG. 3 is a diagrammatic side cross sectional view of an alternative form of the component of FIG. 2;

FIG. 4 is a diagrammatic exploded detail view of an embodiment of the implement of FIG. 1;

FIG. 5 is a diagrammatic side cross sectional view of a further embodiment of the implement of FIG. 1;

FIG. 6 is a diagrammatic side cross sectional view of still another embodiment of the implement of FIG. 1;

FIG. 7 is a diagrammatic view of components of a kit including any of the embodiments of the implement of FIG. 1 and other components;

FIG. 8 is a diagrammatic view of an implement for cleaning ears according to another aspect of the invention, shown in a first position;

FIG. 9 is a diagrammatic view of an implement for cleaning ears in FIG. 8 shown in a second position;

FIG. 10 is a diagrammatic view of an implement for cleaning ears according to another aspect of the invention, shown in a first position; and

FIG. 11 is a diagrammatic view of an implement for cleaning ears in FIG. 10 shown in a second position.

### DETAILED DESCRIPTION

Referring first to FIG. 1, according to at least one aspect of the invention, there is shown an implement 100 for cleaning ears (not shown). Implement 100 may comprise an elongated stem 102 having a proximal end 104 and a distal end 106, and a flexible cleaning head 108 surrounding and coupled to distal end 106 of elongated stem 102. In the embodiment of FIG. 1, elongated stem 102 serves as a handle, and may be curved or contoured as shown to enhance grip characteristics. Therefore, elongated stem 102 may have different transverse dimensions 112 at a handle portion 114 thereof.

It should be noted that proximal and distal ends 104, 106 are semantic devices of convenience, and should not be interpreted narrowly as indicating only the very tips of elongated stem 102. Rather, the term “end” should be understood to encompass some of the length of elongated stem 102.

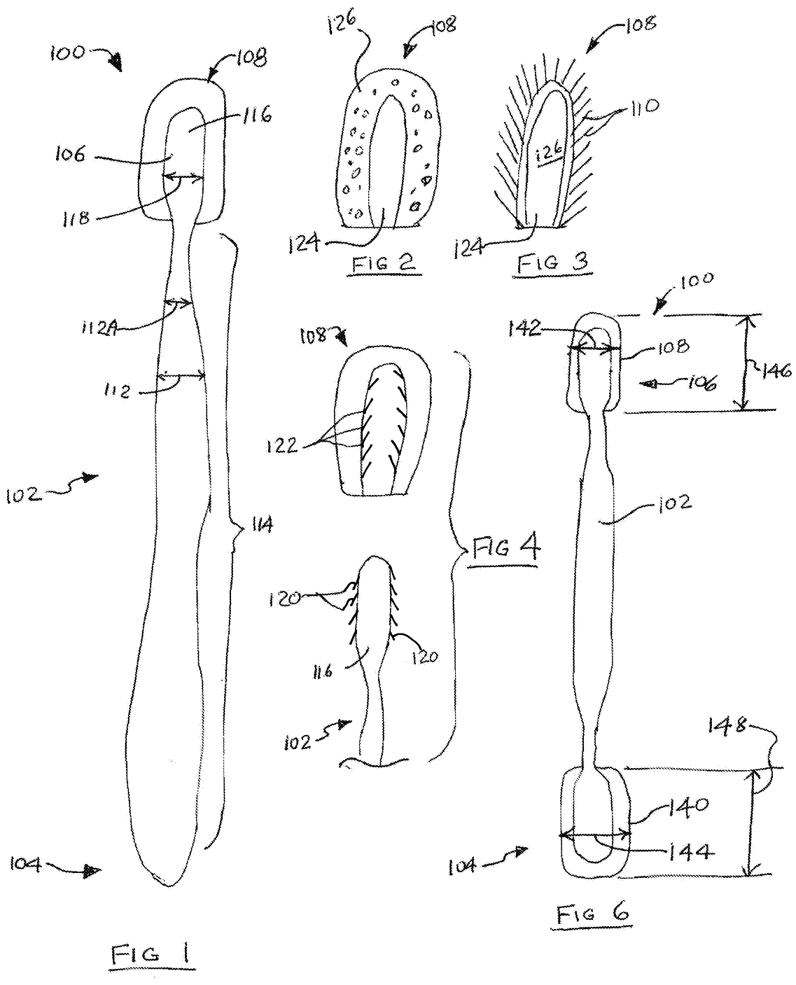

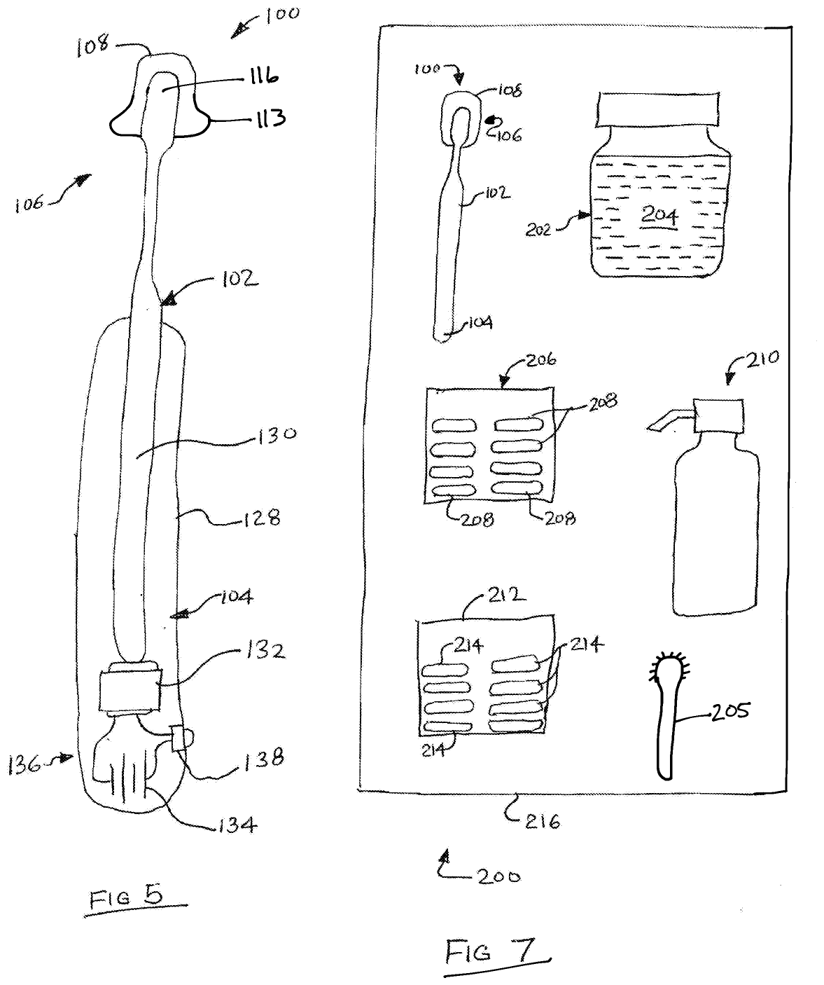

Shown only generically in FIG. 1, cleaning head 108 may comprise an open cell foam, bristle, cloth or any fabric (FIG. 2). Alternatively, or optionally in addition to the open cell foam, cleaning head 108 may comprise bristles 110 (FIG. 3) projecting outwardly from a hollow base member 126. Hollow base member 126 is a structural member for engaging enlarged head 116 of elongated stem 102, for receiving therapeutic fluids for subsequent application to the ear, and to support second teeth 122 where the latter are used. In the embodiment of FIG. 2, hollow base member 126 comprises open cell foam. FIGS. 2 and 3 illustrate a cavity 124 which may be formed within cleaning head 108 to accommodate insertion of enlarged head 116 of elongated stem 102.

Unless otherwise indicated, the terms “first”, “second”, etc., are used herein merely as labels, and are not intended to impose ordinal, positional, or hierarchical requirements on the items to which these terms refer. Moreover, reference to, e.g., a “second” item does not either require or preclude the existence of, e.g., a “first” or lower-numbered item, and/or, e.g., a “third” or higher-numbered item.

Cleaning head 108 may be adhered or configured to removably engage distal end 106 of elongated stem 102. Where cleaning head 108 is configured to removably engage distal end 106 of elongated stem 102 by elastic contraction thereover. This may be a consequence of elastic properties of the open cell foam, the latter being fabricated from a synthetic polymer or from natural sponge material.

In implement 100, elongated stem 102 may have handle portion 114 having transverse dimensions (e.g., as indicated by reference numeral 112), and distal end 106 of elongated stem 102 may comprise an enlarged head 116 having a transverse dimension 118 greater than that of handle portion 114 of elongated stem 102. Of course, handle portion 114 may have transverse dimensions 112 greater in magnitude than transverse dimension 118 of enlarged head 116. In the latter case, elongated stem 102 will have a neck of transverse dimension (e.g., 112A) smaller than either another transverse dimension 112 of elongated stem 102 and enlarged head 116. Enlarged head 116 improves grasp of elongated stem 102 by cleaning head 108.

In an embodiment shown in FIG. 4, cleaning head 108 is configured to removably engage elongated stem 102 by interengagement of respective first teeth 120 of elongated stem 102 and second teeth 122 of cleaning head 108. Second teeth First and second teeth 120, 122 may be spines or bristles formed integrally with respective elongated stem 102 and cleaning head 108, for example, and may be sufficiently flexible to bend and conform to one another as cleaning head 108 is installed over enlarged head 116. Bending and conforming will contribute to interengagement of teeth 120 and 122 to a degree enabling retention of cleaning head 108 on enlarged head 116 of elongated stem 102.

In the embodiment of FIG. 4, first teeth 120 project away from elongated stem 102 and second teeth 122 project toward elongated stem 102 when cleaning head 108 is installed on elongated stem 102, as seen in FIG. 1.

In an alternative to removable cleaning head 108 of FIGS. 2 and 3, cleaning head 108 may be permanently coupled to distal end 106 of elongated stem 102. This may be accomplished by use of adhesive (not shown), by fusion bonding cleaning head 108 to elongated stem 102, or in any other suitable way.

Turning now to FIG. 5, another embodiment is described wherein implement 100 may further comprise a handle 128 surrounding a proximal end portion 130 of elongated stem 102. Elongated stem 102 is rotatable relative to handle 128. Handle 128 includes a motor 132 rotatably drivingly connected to elongated stem 102, and a battery 134 operably connected to motor 132 to provide power to motor 132. Circuitry 136 (indicated in a single line as segments separated by other electrical components) connects motor 132 to battery 134 and includes an on-off switch 138 controlling operation of motor 132. On-off switch 138 may also control rotational speed of motor 132. Rotation of elongated stem 102 enables cleaning head 108 to expeditiously apply therapeutic fluids to the ear canal (not shown), to extract wax and dirt from the ear canal, or both. Vibratory motion of elongated stem 102 is also contemplated as a means to expeditiously apply therapeutic fluids to the ear canal (not shown), to extract wax and dirt from the ear canal, or both. Note that cleaning head 108 in FIG. 5 shows that the cleaning head may include a stop 113 that may be formed, molded or added to the cleaning head 108. Stop 113 is a flared portion that prevents the cleaning head 108 from being inserted too deeply into the ear canal by the user an can be added to in any of the embodiments shown herein.

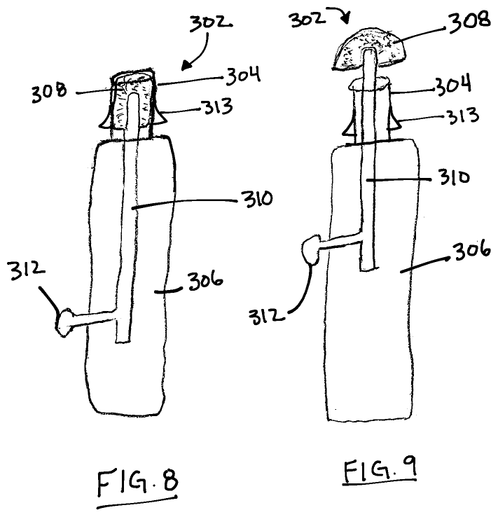

Turning now to FIG. 6, there is shown an embodiment of implement 100 wherein flexible cleaning head 108 is a first flexible cleaning head, and implement 100 further comprises a second cleaning head 140 at proximal end 104 of elongated stem 102. First cleaning head 108 has first surface characteristics and second cleaning head 140 has second surface characteristics different from the first surface characteristics of first flexible cleaning head 108. This enables implement 100 of FIG. 6 to accommodate a wider variety of scrubbing and cleaning techniques than that of FIGS. 1 and 5.

In implement 100 of FIG. 6, the first surface characteristics may include a first transverse dimension 142, and the second surface characteristics may include a second transverse dimension 144 different from first transverse dimension 142. Independently of transverse dimensions 142 and 144, the first surface characteristics may include a first length, width and shape 146, and the second surface characteristics may include a second length, width and shape 148 different from first length, width and shape 146. The shapes and contours of first and second cleaning heads 108 and 140 may also differ from each other. Still other characteristics may differ between first and second cleaning heads 108 and 140. For example, first and second cleaning heads 108 and 140 may both comprise open cell foam, but have different pore sizes. In a further example, first and second cleaning heads 108 and 140 may both have bristles 110, but of different lengths, flexibility, and/or thicknesses.

The first surface characteristics may include a first matter retainer (e.g. bristles) and the second surface characteristics may include a second matter retainer (e.g. bristles of different diameter, stiffness, or spacing) different from the first matter retainer. The first matter retainer may comprise open cell foam of one pore size or flexibility, while the second matter retainer may have different pore size or flexibility. A matter retainer is a cleaning head 108 (or 140) having structure for trapping ear wax, dirt, and other solid matter in the ear and retaining the solid matter while withdrawing implement 100 from the ear.

Exemplary dimensions of implement 100 include an overall length (from proximal end 104 to distal end 106) of seven to eight inches. Transverse dimensions of elongated stem may range from three sixteenths of an inch to three quarters of an inch. Wall thickness of cleaning head 108, where comprising open cell foam (FIG. 2), may range from one eighth of an inch to three eighths of an inch, and may vary within this range at different degrees of rotation about the length (i.e., the front view may show different wall thicknesses from a side view). Where used, bristles 110 may vary similarly. Enlarged head 116 may vary in transverse dimensions as well. Exemplary dimensions reflect preferred embodiments, but do not limit dimensional possibilities.

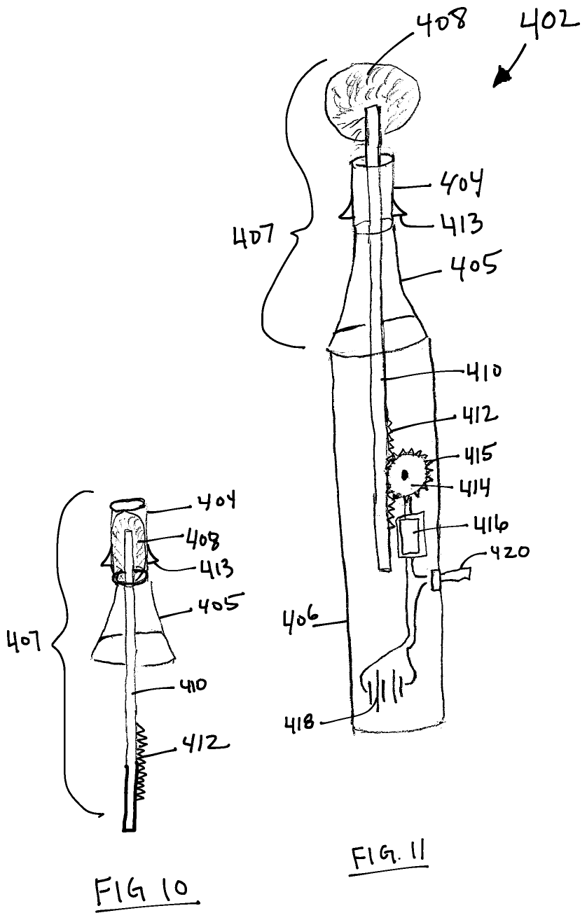

Turning now to FIGS. 8 and 9, presented therein is an additional embodiment of an implement for cleaning ears 302. In this variation, flexible tube 304, preferably made of silicon, is mounted on a body that acts as a handle 306. In FIG. 8, it can be seen that the flexible tube 304 houses and compresses a cleaning head 308 in an insertion position and is utilized to provide smooth insertion into the inner ear canal without pushing wax and/or dirt in. Once the flexible tube is inserted into the ear canal, the cleaning head 308 is then advanced out of the flexible tube to an extraction position in the ear canal. In the extraction position shown in FIG. 9, the cleaning head 308 expands to allow for the cleaning of the ear canal as the cleaning head is pulled out of the ear canal. FIGS. 8 and 9 depict a manual embodiment of the implement for cleaning ears 302. In this embodiment, the operation of the cleaning head in and out of the flexible tube 304, is provided by rod 310 that is fixed to the cleaning head 308 and extends into the handle 306. Movement of the rod 310 is provided by finger slide 312 that is functionally connected to rod 310. Finger slide 312 is retained in a channel (not shown) that includes stops at each travel extreme such that the travel of the rod 310 is confined between the two extremes to avoid potential injury to the user. In addition, flexible tube 304 may include a stop 313 that may be molded in the tube or added to the tube. Stop 313 is a flared portion that prevents the tube 304 from being inserted too deeply into the ear canal by the user.

Turning now to FIGS. 10 and 11, presented therein is an additional embodiment of an implement for cleaning ears 402. Again, in this variation a flexible tube 404, preferably made of silicon, is mounted on a stem 405 which detachably connects to a body that acts as a handle 406. In FIG. 10, it can be seen that the flexible tube 404 houses and compresses a cleaning head 408 in an insertion position and is utilized to provide smooth insertion into the inner ear canal without pushing wax and/or dirt in.

Shown in FIG. 10 is the detachable component 407 of ear cleaning implement 402 that includes the flexible tube 404, the stem 405, and a compressed cleaning head 408 that is fixed to rod 410. As can be see, rod 410 includes a teethed portion 412.

In the extraction position shown in FIG. 11, the cleaning head 408 expands to allow for the cleaning of the ear canal as the cleaning head is pulled out of the ear canal. FIGS. 10 and 11 depict an electronic/motorized embodiment of the implement for cleaning ears 402. In this embodiment, the operation of the cleaning head in and out of the flexible tube 404, is provided by rod 410 that is fixed to the cleaning head 408 and extends into the handle 406. Movement of the rod 410 is provided by gear 414 that is rotated by motor 416 that is powered by an energy source 418 (ex. battery). Gear 414 includes a teethed surface 415 that matingly engages with the teethed portion 412 of the rod 410. A toggle switch 420 is electronically connected to the energy source 418 and motor 416 such that the user can control the movement of the cleaning head 408 in and out of the flexible tube 404. Note that the ear cleaning implement 402 may include structural or electronically managed stops (not shown) that includes stops at each travel extreme such that the travel of the rod 410 is confined between the two extremes to avoid potential injury to the user. In addition, flexible tube 404 may include a stop 413 that may be molded in the tube or added to the tube. Stop 413 is a flared portion that prevents the tube 404 from being inserted too deeply into the ear canal by the user.

Referring primarily to FIG. 7 but also to FIGS. 1-6 and FIGS. 8-11, the invention may be practiced as a kit 200 for cleaning the ears.

EAR SPONGE—Kit 200 may comprise implement 100 (302 or 402) for cleaning ears, implement 100 comprising elongated stem 102 having proximal end 104 and distal end 106, and a flexible liquid permeable cleaning head (e.g., cleaning head 108) surrounding and coupled to distal end 106 of elongated stem 102.

EAR FOAM—Kit 200 may also include a container 202 containing any cleansing/therapeutic agents—gel, paste, liquid, cream, mousse or oil foam 204, oil foam 204 comprising oil, soap, for use with or without and water (ingredients not separately shown). Treating flaky ears, swimmer ear, ear pimples

MOISTURIZING DEODERIZING EAR MYST—Kit 200 may also include a spray container 210 containing antibacterial moisturizing deodorizing and therapeutic liquid (not separately shown).

EAR SCRUB WIPES—Kit 200 may also include a package 206 containing a supply of first liquid permeable pads 208 impregnated with antibacterial and therapeutic liquid (not separately shown).

MOISTURIZING DEODERIZING EAR WIPES—Optionally, kit 200 may further comprise a container 212 containing a supply of second liquid permeable pads 214 impregnated with an astringent deodorizing agent (not separately shown).

EAR WIPE HANDLE—Also a hand-held implement 205 for wipes. Having textured teeth or Velcro head so wipes can stick on it rather than the user using their fingers to hold the wipes.

As employed herein, the term “supply” is to be interpreted as a plurality of pads 208 or 214 in a number suitable for consumer use by one person, such as two to fifty pads 208 or 214 in the course of routine retail sales for example. It should be recognized that kit 200 may be intended for use with plural patients, e.g., by a health facility or by health personnel responding to a local emergency event, and could therefore comprise more than fifty pads 208 or 214.

Kit 200 may include the following therapeutic liquids mentioned above. Antibacterial liquid may comprise in an aqueous or oil base, bacitracin, neomycin, other known bacteriocidal agents, and combinations of these. The astringent agent may comprise witch hazel moisturizing glycerin deodorizing essential oils for example.

Components of kit 200 described above are enclosed within a common enclosure 216. The latter may be any type of packaging suitable for shipping or for retail and capable of protecting components of kit 200 from casual damage and contamination, for example.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. An implement for cleaning ears, the implement comprising an elongated stem having a proximal end and a distal end, and a flexible cleaning head surrounding and coupled to the distal end of the elongated stem.

2. The implement of claim 1, wherein the flexible cleaning head comprises an open cell foam.

3. The implement of claim 1, wherein the flexible cleaning head is selected from the group consisting of bristles, a sponge and cloth.

4. The implement of claim 1, wherein the flexible cleaning head is configured to removably engage the distal end of the elongated stem.

5. The implement of claim 4, wherein the flexible cleaning head is configured to removably engage the distal end of the elongated stem by elastic contraction thereover.

6. The implement of claim 5, wherein the elongated stem has a handle portion having a transverse dimension, and the distal end of the elongated stem comprises an enlarged head having a transverse dimension greater than that of the handle portion of the elongated stem.

7. The implement of claim 2, wherein the flexible cleaning head is configured to removably engage the elongated stem by interengagement of respective first teeth of the elongated stem and second teeth of the flexible cleaning head.

8. The implement of claim 7, wherein the first teeth project away from the elongated stem and the second teeth project toward the elongated stem when the flexible cleaning head is installed on the elongated stem.

9. The implement of claim 7, wherein the flexible cleaning head is permanently coupled to the distal end of the elongated stem.

10. The implement of claim 7, wherein the elongated stem has a handle portion having a transverse dimension, and the distal end of the elongated stem comprises an enlarged head having a transverse dimension greater than that of the handle portion of the elongated stem.

11. The implement of claim 1, further comprising a handle surrounding a proximal end portion of the elongated stem, wherein the elongated stem is rotatable relative to the handle, and the handle includes a motor rotatably drivingly connected to the elongated stem, and a battery operably connected to the motor to provide power to the motor.

12. The implement of claim 1, wherein
the flexible cleaning head is a first flexible cleaning head,
the implement further comprises a second flexible cleaning head at the proximal end of the elongated stem, and
the first flexible cleaning head has first surface characteristics and the second flexible cleaning head has second surface characteristics different from the first surface characteristics of the first flexible cleaning head.

13. The implement of claim 12, wherein the first surface characteristics include a first transverse dimension, and the second surface characteristics include a second transverse dimension different from the first transverse dimension.

14. The implement of claim 12, wherein the first surface characteristics include a first length, and the second surface characteristics include a second length different from the first length.

15. The implement of claim 12, wherein the first surface characteristics include a first matter retainer (e.g. bristles) and the second surface characteristics include a second matter retainer (e.g. bristles of different diameter, stiffness, or spacing; pores of different pore size) different from the first matter retainer.

16. An implement for cleaning ears, the implement comprising:
a cleaning head;
a flexible tube mounted to a body; and
means to advance the cleaning head from a stored position in the flexible tube to an extraction position out from the flexible tube.

17. The implement of claim 16, wherein the means to advance the cleaning head is non-electrical.

18. The implement of claim 16, wherein the means to advance the cleaning head is electrical.

19. A kit for cleaning the ears, the kit comprising:
an implement for cleaning ears, the implement comprising an elongated stem having a proximal end and a distal end, and a flexible liquid permeable cleaning head surrounding and coupled to the distal end of the elongated stem;
a container containing oil foam, liquid soap, gel, paste the oil foam comprising oil, soap, and water;
a package containing a supply of liquid permeable pads impregnated with antibacterial liquid; and
a spray container containing antibacterial moisturizing deodorizing liquid.

20. The kit of claim 19, further comprising a container containing a supply of liquid permeable pads impregnated with an astringent agent.
